(19) United States
(12) Patent Application Publication
(10) Pub. No.: US 2023/0364576 A1
(43) Pub. Date: Nov. 16, 2023

(54) KRAFT LIGNIN NANOPARTICLES

(71) Applicant: LUXEMBOURG INSTITUTE OF SCIENCE AND TECHNOLOGY (LIST), Esch-sur-Alzette (LU)

(72) Inventors: Ahilan MANISEKARAN, Esch-sur-Alzette (LU); Jean-Sébastien THOMANN, Esch-sur-Alzette (LU)

(21) Appl. No.: 18/030,488

(22) PCT Filed: Oct. 1, 2021
(86) PCT No.: PCT/EP2021/077055

§ 371 Date: Apr. 5, 2023

(30) Foreign Application Priority Data
Oct. 5, 2020 (LU) LU102105

(51) Int. Cl. B01J 13/00; C08L 97/00; C08J 3/14

(52) U.S. Cl. CPC B01J 13/0021; B01J 13/0086; C08J 3/14; C08L 97/005; C08J 2397/00

(57) ABSTRACT

The disclosure relates to a method for manufacturing a colloidal dispersion of Kraft lignin (KL) nanoparticles, said method comprising the steps of (a) providing KL; (b) dissolving said KL into a solvent, to obtain a solution having a concentration of Kraft lignin of at least 15 mg/ml; and (c) mixing said solution with an antisolvent under mixing conditions, to provide a colloidal dispersion of nanoparticles. Said method is remarkable in that the solvent used in step (b) of dissolving said KL is one or more organic solvents, and in that step (c) of mixing is performed by the addition of the solution of step (b) into an antisolvent being or comprising water. The disclosure also relates to spherical KL nanoparticles with an average diameter size ranging from 9 nm up to 70 nm. The disclosure further relates to various uses of said spherical KL nanoparticle.

### FIELD OF THE DISCLOSURE

The present disclosure relates to a method for manufacturing a colloidal dispersion of Kraft lignin nanoparticles. Conditions to obtain dried Kraft lignin nanoparticles are also described.

### BACKGROUND OF THE DISCLOSURE

Different methods exist to develop lignin nanoparticles and more specifically Kraft lignin (KL) nanoparticles. Thus, solvent-shifting, pH-shifting, crosslinking/polymerization, mechanical treatment, ice-segregation, template-based synthesis, aerosol processing, electrospinning and/or use of carbon dioxide as anti-solvent have been carried out (see study entitled “Lignin from micro- to nanosize: production methods” from Beisl S. et al. (Int. J. Mol. Sci., 2017, 18, 1244). Among these methods, the solvent-shifting method is a potential green chemistry method that is based on dissolving the lignin into a solvent and incorporating an anti-solvent in the solution that has been formed under different conditions. Thus, nanoparticles are generated due to their decreasing solubility in the medium.

In WO 2019/081819, the solvent-shifting method has been applied to form colloidal lignin particles. It comprises a step of dissolving lignin in a mixture of both a solvent and a co-solvent. Once the lignin is dissolved, a step of feeding said solution into an anti-solvent is carried out. Colloidal KL particles of generally spherical shape are thus obtained with an average diameter that is below 400 nm, for example 230 nm. It is described that such colloidal particles can further be functionalized with phenol-formaldehyde to increase the reactivity of the surface.

In the study entitled “Scaling up production of colloidal lignin particles”, from Leskinen T. et al. (Nordic Pulp & Paper Research Journal, 2017, 32 (4), 583-593), an aqueous dispersion of colloidal lignin particles has been prepared by dissolving KL in a THF-water mixture, having a ratio THF/water of at least 3/1. Magnetic stirring was applied to fully dissolve the lignin in the solvent mixture. Spherical lignin particles with an average diameter of 220 nm could be produced.

In the study entitled “A simple process for lignin nanoparticle preparation”, from Lievonen M. et al. (Green Chem., 2016, 18, 1416-1422), spherical KL nanoparticles with a diameter superior to 200 nm and up to 3000 nm have been developed. KL was dissolved in THF, introduced in a dialysis bag (i.e. permeable membrane) and then immersed in excess of deionized water. Lignin nanoparticles were formed during the dialysis process, which took place for at least 24 hours under slow stirring. Dialysis allows the exchange of solvent through the permeable membrane. Water is thus incorporated into the dialysis bag allowing KL nanoparticles to form while THF is conducted out of the dialysis bag. The lowest diameter can be obtained when the KL concentration is about 1 mg/ml before the dialysis. At 20 mg/ml of lignin concentration before the dialysis, the dispersion has become unstable and particles with a size superior to 1000 nm are obtained.

In the study entitled “Assessing the potential of lignin nanoparticles as drug carrier: synthesis, cytotoxicity and genotoxicity studies”, from Siddiqui L., et al., (Int. J. of Bio. Macromol., 2020, 152, 786-802), DMSO was used to dissolved the Kraft lignin at a concentration of 5 mg/ml, then deionized water was used as a dialysis medium and the dialysis was performed for at least 24 hours under continuous magnetic stirring for the formation of nanoparticles and removal of DMSO. The obtained nanoparticles show a particle size of about 120 nm with a polydispersity index ranging from 0.1 to 0.3. However, although being of small size, the obtained nanoparticles are not uniform in shape and size, and unorganized structures can be seen in the background of the nanoparticles, since they were synthesized by a dialysis method. If however, hydrochloric acid is added, upon dialysis for at least 24 h, bigger non-spherical nanoparticles of a size of about 200 nm are obtained. Upon self-assembling of the lignin molecules in hydrochloric acid in a first step lasting 4 hours followed by a second step of dialysis lasting 20 hours, it was possible to obtain uniform spherical nanoparticles of a size of about 140 nm.

In the study entitled “Synthesis and characterization of biodegradable lignin nanoparticles with tunable surface properties”, from Richter A. P., et al. (Langmuir, 2016, 32, 6468-6477), KL nanoparticles with a size ranging between 50 nm and 200 nm have been developed by adding nitric acid into a mixture of lignin and ethylene glycol. Transmission Electron Microscopy (TEM) analysis of such nanoparticles revealed that they were irregularly shaped. Such particles are nevertheless interesting since the available surface area is greater than if the particles were spherical.

In the study entitled “Green synthesis of lignin nanoparticle in aqueous hydrotropic solution toward broadening the window for its processing and application”, from Chen L., et al. (Chem. Eng. J., 2018, 346, 217-225), lignin nanoparticles are formed by self-assembly of alkaline lignin diluted beforehand in aqueous hydrochloric acid solution, in the recyclable and non-toxic aqueous sodium p-toluenesulfonate solution at room temperature.

In the study entitled “One pot synthesis of environmentally friendly lignin nanoparticles with compressed liquid carbon dioxide as an antisolvent”, from Myint A. A., et al., (Green Chem., 2016, 18, 2129-2146), quasi-spherical KL nanoparticles with a mean particle diameter of 38 nm were obtained by using precipitation with compressed antisolvent (PCA). KL is dissolved into DMF and the antisolvent chosen is compressed liquid CO₂. Depending on the applied conditions (temperature, pressure, initial lignin concentration, lignin solution flow rate, CO₂ density and CO₂ mole fraction), quasi-spherical nanosized KL particles with a maximum mean diameter smaller than 80 nm were obtained. Such KL nanoparticles are qualified as more or less aggregated and/or coalesced. Indeed, High-Resolution Transmission Electron Microscopy (HRTEM) images allow observing that these KL nanoparticles are inerratic nanoparticles due to the diffusion effect coming from the utilization of compressed liquid CO₂. Such fused nanoparticles lead therefore to nanoparticles with poor dispersion properties and also poor optical transparency.

The patent application WO 2020/109671 discloses a method of preparing a dispersion of colloidal lignin by providing a solution of lignin in a mixture of an organic solvent and a non-solvent and by increasing the ratio of the non-solvent to the organic solvent, notably by evaporating the organic solvent, to produce an aqueous dispersion of colloidal lignin particles. By increasing the concentration of lignin into the solution, it is described that larger particles are obtained.

However, for more advanced applications, there is still the need to provide lignin nanoparticles that are unfused, spherical and present a very small size, such as below 200 nm, or preferably below 150 nm, more preferably below 100 nm, even more preferably below 70 nm.

### SUMMARY OF THE DISCLOSURE

According to a first aspect, the disclosure provides a method for manufacturing a colloidal dispersion of Kraft lignin nanoparticles, said method is remarkable in that it comprises the following steps:
a) providing Kraft lignin;
b) dissolving said Kraft lignin into a solvent, to obtain a solution having a concentration of Kraft lignin of at least 15 mg/ml;
c) mixing the solution of step (b) with an antisolvent under mixing conditions, to provide a colloidal dispersion of nanoparticles;
wherein the solvent used in step (b) of dissolving said Kraft lignin is one or more organic solvents, and wherein step (c) of mixing is performed by the addition of the solution of step (b) into an antisolvent being or comprising water.

It is particularly preferred that according to this first aspect, the disclosure provides a method for manufacturing a colloidal dispersion of Kraft lignin nanoparticles having an average diameter size ranging between 9 nm and 200 nm, said method is remarkable in that it comprises the following steps:
a) providing Kraft lignin;
b) dissolving said Kraft lignin into a solvent, to obtain a solution having a concentration of Kraft lignin of at least 15 mg/ml;
c) mixing the solution of step (b) with an antisolvent under mixing conditions, to provide a colloidal dispersion of nanoparticles having a diameter size ranging between 9 nm and 200 nm;
wherein the solvent used in step (b) of dissolving said Kraft lignin is one or more organic solvents, and wherein step (c) of mixing is performed by the addition of the solution of step (b) into an antisolvent being or comprising water.

For example, the average diameter size is ranging from 15 nm, or even from 9 nm, and no more than 200 nm as determined by imaging techniques, preferably ranging from 15 nm, or even from 9 nm, and no more than 150 nm, preferably no more than 100 nm and even more preferably no more than 70 nm.

For example, the solvent used in step (b) of dissolving said Kraft lignin is a single organic solvent.

Advantageously, the method of the first aspect of the disclosure is devoid of dialysis step.

Surprisingly, the inventors have found that it was possible to form spherical KL nanoparticles having a size below 200 nm, preferably below 150 nm, more preferably below 100 nm, even more preferably below 70 nm, by dissolving a large amount of KL into one or more organic solvents (for example into only one organic solvent) which results in a high concentration of such KL into said organic solvent(s) and adding the solution to an anti-solvent, which is or comprise water.

Self-assembly of lignin nanoparticles was found to be favoured due to the presence of π-π stacking, van der Waals interactions, hydrogen bonding and electrostatic repulsions between the aromatic structures of lignin. Coalescence and Ostwald ripening are the main effects behind the growth of nanoparticles from nucleation to the final packed polymer nanoparticles. The addition of the KL solution onto the water is thought to provide a huge free space to form the KL nanoparticles, which means that it facilitates the formation of a large number of small nuclei of KL nanoparticles. Since water has a lower affinity towards lignin, it acts as a quenching medium. Once the addition is completed, water completely quenches the growth of KL nanoparticles. The reduction of the concentration of the one or more organic solvents in the medium, or in other words the high concentration of KL into said organic solvent(s) does prevent the phenomenon of coalescence and Ostwald ripening the systems and leads to the production of small nanoparticles, i.e. with an average diameter size ranging from 15 nm, or even from 9 nm, and no more than 200 nm as determined by imaging techniques, preferably ranging from 15 nm, or even from 9 nm, and no more than 150 nm, preferably no more than 100 nm and even more preferably no more than 70 nm; and with a well-distinguishable morphology, also as determined by imaging techniques such as Scanning Electron Microscopy for the nanoparticles presenting a bigger size and Helium Ion Microscopy for the nanoparticles presenting a smaller size.

The method found by the inventors also provides several other advantages. Since flammable organic solvents are only used to dissolve KL, the amount of such solvents is drastically reduced, which facilitates safety in handling and processing the protocols in industries. The use of the green antisolvent, namely water, is practical since the water can be recycled and reused. In addition, the use of a higher concentration of KL allows for obtaining a high yield of KL nanoparticles. This facilitates industrial up-scalability of the process.

With preference, the solution provided at step (b) has a concentration of Kraft lignin of at least 17 mg/ml, more preferably of at least 20 mg/ml, even more preferably of at least 25 mg/ml.

Advantageously, the mixing step (c) is performed during a period ranging between 45 seconds and 1 hour, or between 45 seconds and 30 minutes, or between 45 seconds and 15 minutes.

Advantageously, steps (b) and (c) are performed in a single reactor.

### Organic Solvents With a Partition Coefficient Inferior to -0.50

In a more preferred embodiment, the one or more organic solvents have a partition coefficient inferior to -0.50 and/or a dipole moment of at least 3 D.

With preference, one or more of the following features can be used to better define said more preferred embodiment of the disclosure:
The one or more organic solvents are selected from dimethyl sulfoxide (DMSO), dimethylformamide (DMF) and any mixture thereof; with preference, the one or more organic solvents are or comprise dimethyl sulfoxide (DMSO).
The one or more organic solvents are dried before the implementation of step (b).
The solution of step (b) has a concentration of Kraft lignin ranging between 15 mg/ml and 35 mg/ml, preferentially between 17 mg/ml and 33 mg/ml, more preferentially between 20 mg/l and 30 mg/ml.

With preference, one or more of the following features can be used to better define the mixing conditions of step (c):
The volume ratio between the antisolvent and the solution of step (b) is ranging between 0.5 and 2.5, preferentially between 0.6 and 2.5.
The mixing is performed at a temperature ranging from 1° C. to 90° C., preferentially ranging from 5° C. to 85° C., more preferentially from 10° C. to 80° C., even more preferentially from 15° C. to 75° C.
The mixing is performed under stirring at a stirring speed ranging between 500 rpm and 2500 rpm, preferentially between 600 rpm and 2400 rpm, more preferentially between 700 rpm and 2300 rpm, even more preferentially between 800 rpm and 2200 rpm.
The mixing is performed under inert atmosphere and under stirring at a stirring speed superior to 2500 rpm, preferentially superior to 2600 rpm, more preferentially superior to 2700 rpm, even more preferentially superior to 2800 rpm.

In a more preferred embodiment, the disclosure provides a method for manufacturing a colloidal dispersion of Kraft lignin nanoparticles, said method comprising the following steps:
a) providing Kraft lignin;
b) dissolving said Kraft lignin into a solvent, to obtain a solution having a concentration of Kraft lignin of at least 15 mg/ml;
c) mixing the solution of step (b) with an antisolvent under mixing conditions, to provide a colloidal dispersion of nanoparticles;
said method is remarkable in that the solvent used in step (b) of dissolving said Kraft lignin is one or more organic solvents having a partition coefficient inferior to -0.50, the step (c) of mixing is performed by the addition of the solution of step (b) into water; and one or more of the following features are true, preferably all the following features are true:
the one or more organic solvents are or comprise dimethyl sulfoxide (DMSO); and/or
the concentration of Kraft lignin into the solution of step (b) is ranging between 15 mg/ml and 35 mg/ml; and/or
the step (c) of mixing is performed by the addition of the solution of step (b) into water at a volume ratio between the water and the solution of step (b) of at least 0.5; and/or
the step (c) of mixing is performed at a stirring speed ranging between 500 rpm and 2500 rpm; and/or
the temperature of step (c) is ranging between 1° C. and 90° C.

### Organic Solvents With a Partition Coefficient Superior to -0.50

In a preferred embodiment, said organic solvent has a partition coefficient superior to -0.50 and/or a dipole moment inferior to 3 D.

With preference, one or more of the following features can be used to better define said preferred embodiment of the disclosure:
The one or more organic solvents are selected from 1,4-dioxane, dichloromethane, tetrahydrofuran (THF), ethyl acetate, acetone, and any mixture thereof; with preference, one or more organic solvents are or comprise tetrahydrofuran (THF).
The one or more organic solvents are dried before the implementation of step (b).
The solution of step (b) has a concentration of Kraft lignin ranging between 15 mg/ml and 55 mg/ml, preferentially between 17 mg/ml and 53 mg/ml, more preferentially between 20 mg/ml and 50 mg/ml.

With preference, one or more of the following features can be used to better define the mixing conditions of step (c):
The volume ratio between the antisolvent and the solution of step (b) is ranging between 0.1 and 2.5, preferentially between 0.2 and 2.4, more preferentially between 0.3 and 2.2, even more preferentially between 0.4 and 2.1.
The mixing is performed at a temperature ranging from 1° C. to 65° C., preferentially between 5° C. and 60° C., more preferentially between 10° C. and 55° C., even more preferentially between 15° C. and 50° C.
The mixing is performed under stirring at a stirring speed ranging between 300 rpm and 2500 rpm, preferentially between 350 rpm and 2400 rpm, more preferentially between 400 rpm and 2200 rpm, even more preferentially between 500 rpm and 2000 rpm.
The mixing is performed under inert atmosphere and under stirring at a stirring speed superior to 2500 rpm, preferentially superior to 2600 rpm, more preferentially superior to 2700 rpm, even more preferentially superior to 2800 rpm.

In a preferred embodiment, the disclosure provides a method for manufacturing a colloidal dispersion of Kraft lignin nanoparticles, said method comprising the following steps:
a) providing Kraft lignin;
b) dissolving said Kraft lignin into a solvent, to obtain a solution having a concentration of Kraft lignin of at least 15 mg/ml;
c) mixing the solution of step (b) with an antisolvent under mixing conditions, to provide a colloidal dispersion of nanoparticles;
said method is remarkable in that the solvent used in step (b) of dissolving said Kraft lignin is one or more organic solvents having a partition coefficient superior to -0.50, the step (c) of mixing is performed by the addition ofthe solution of step (b) into water; and at least one or all of the following features is true, preferably all the following features are true:
the single organic solvent is tetrahydrofuran (THF); and/or
the concentration of Kraft lignin into the solution of step (b) is ranging between 15 mg/ml and 55 mg/ml; and/or
the step (c) of mixing is performed by the addition of the solution of step (b) into water at a volume ratio between the water and the solution of step (b) is ranging between 0.1 and 2.5; and/or
the step (c) of mixing is performed at a stirring speed ranging between 300 rpm and 2500 rpm; and/or
the temperature of step (c) is ranging between 1° C. and 65° C.

According to a second aspect, the disclosure provides a method for manufacturing lignin nanoparticles, said method comprising the method for manufacturing a colloidal dispersion of KL nanoparticles according to the first aspect, said method for manufacturing lignin nanoparticles being remarkable in that step (c) is followed by the step (d) of removing said one or more organic solvents, preferentially by evaporation with pressured controlled rotary evaporator and/or by dialysis.

It is to be highlighted that the dialysis is performed only to remove the one or more organic solvents, especially the one or those with high boiling points. In any case, the dialysis used in the present method does not assemble the Kraft lignin into KL nanoparticles.

With preference, said step (d) is followed by a step (e) of freeze-drying. More preferably, said step (e) of freeze-drying is carried out at a temperature ranging between -50° C. and -100° C., preferentially between -55° C. and -95° C.

Advantageously, said step (e) of freeze-drying is carried out at a pressure ranging between 0.05 Pa and 0.20 Pa, preferentially between 0.06 Pa and 0.19 Pa.

According to a third aspect, the disclosure provides Kraft lignin nanoparticle, said nanoparticle being remarkable in that said nanoparticle is spherical as determined by imaging techniques, in that said nanoparticle has an average diameter size ranging from 15 nm up to 200 nm, or preferably from 15 nm up to 70 nm, as determined by Scanning Electron Microscopy and in that said nanoparticle has a glass transition temperature which is superior to the glass transition temperature of the Kraft lignin as determined by Differential Scanning Calorimetry.

With preference, the disclosure provides Kraft lignin nanoparticle, said nanoparticle being remarkable in that said nanoparticle is spherical as determined by imaging techniques, in that said nanoparticle has an average diameter size ranging from 9 nm up to 70 nm as determined by imaging techniques and in that said nanoparticle has a glass transition temperature which is superior to the glass transition temperature of the Kraft lignin as determined by Differential Scanning Calorimetry.

For example, the imaging techniques are Scanning Electron Microscopy (SEM) and/or Helium Ion Microscopy (HIM). SEM can be used for the nanoparticles presenting a bigger average diameter size while HIM is used for the nanoparticles presenting a smaller average diameter size. In general, a size of about 15 nm can be well-detected by SEM. As the KL nanoparticles generated by DMSO have in general a smaller average diameter size than the KL nanoparticles generated with THF, HIM experiments are carried out on the KL nanoparticles generated with DMSO.

For example, said nanoparticle has an average diameter size ranging from 15 nm up to 60 nm as determined by Scanning Electron Microscopy, or from 15 nm up to 55 nm, or from 15 nm up to 50 nm, or from 15 nm up to 45 nm, or from 15 nm up to 40 nm, or from 15 nm up to 35 nm. Advantageously, said nanoparticle having an average diameter size ranging from 15 nm up to 60 nm has a transmittance taken at a wavelength of 600 nm ranging between 40% and 80% as determined by absorption analysis, preferably between 45% and 75%.

With preference, one or more of the following features can be used to better define the Kraft lignin nanoparticle of the third aspect of the disclosure:
The average diameter size, as determined by Scanning Electron Microscopy is at least 15 nm, and at most 200 nm, preferably at most 150 nm, more preferably at most 100 nm, even more preferably at most 70 nm, most preferably at most 65 nm, even most preferably at most 60 nm or at most 50 nm.
The three-dimensional structure of said nanoparticle comprises at least two types of π-π stacking, as determined by UV-Visible analysis.
Said nanoparticle has a polydispersity index ranging between 0.05 and 0.20 as determined by Dynamic Light Scattering method, preferably between 0.07 and 0.18, more preferably between 0.09 and 0.16, even more preferably between 0.11 and 0.14.
The glass transition temperature which is superior to the glass transition temperature of the Kraft lignin as determined by Differential Scanning Calorimetry is of at least 150° C.
The glass transition temperature which is superior to the glass transition temperature of the Kraft lignin as determined by Differential Scanning Calorimetry is a second glass transition temperature and said nanoparticle has a first glass transition temperature has determined by Differential Scanning Calorimetry. For example, said first glass transition temperature is ranging between 110° C. and 130° C. as determined by Differential Scanning Calorimetry.
Said nanoparticle has a Young’s modulus ranging between 1.0 GPa and 6.0 GPa as determined by Atomic Force Microscopy, preferentially between 1.4 GPa and 5.1 GPa, more preferentially between 1.5 GPa and 5.0 GPa.

According to a fourth aspect, the disclosure provides the use of Kraft lignin nanoparticles according to the third aspect of the disclosure as a reinforcing filler material comprised in a polymer nanocomposite.

According to a fifth aspect, the disclosure provides the use of Kraft lignin nanoparticles according to the third aspect of the disclosure as a sunblock agent.

According to a sixth aspect, the disclosure provides the use of Kraft lignin nanoparticles according to the third aspect of the disclosure as a drug carrier.

### DESCRIPTION OF THE FIGURES

FIG. 1: Scheme representing the growth of KL nanoparticles as a function of the space available between the nanoparticles.

FIG. 2: Scheme showing the three types of π-π stacking in organic compounds comprising aromatic structures.

FIG. 3: Dynamic light scattering (DLS) analysis of the KL nanoparticles in view of different solvents.

FIG. 4: DLS analysis of the KL nanoparticles obtained from KL dissolved in THF at different concentrations.

FIG. 5: DLS analysis of the KL nanoparticles obtained from KL dissolved in DMSO at different concentrations.

FIG. 6: Scanning Electron Microscopy (SEM) images of the KL nanoparticles obtained from KL dissolved in THF at 20 mg/mL.

FIG. 7: SEM images of the KL nanoparticles obtained from KL dissolved in THF at 30 mg/mL.

FIG. 8: SEM images of the KL nanoparticles obtained from KL dissolved in THF at 40 mg/mL.

FIG. 9: SEM images of the KL nanoparticles obtained from KL dissolved in THF at 50 mg/mL.

FIG. 10: SEM images of the KL nanoparticles obtained from KL dissolved in THF at 60 mg/mL.

FIG. 11: DLS analysis of the KL nanoparticles obtained from KL dissolved in THF and added on different amounts of MilliQ water.

FIG. 12: DLS analysis of the KL nanoparticles obtained from KL dissolved in DMSO and added on different amounts of MilliQ water.

FIG. 13: SEM images of the KL nanoparticles obtained from KL dissolved in THF at 20 mg/mL and added on 4 ml of MilliQ water.

FIG. 14: SEM images of the KL nanoparticles obtained from KL dissolved in THF at 20 mg/mL and added on 8 ml of MilliQ water.

FIG. 15: SEM images of the KL nanoparticles obtained from KL dissolved in THF at 20 mg/mL and added on 16 ml of MilliQ water.

FIG. 16: SEM images of the KL nanoparticles obtained from KL dissolved in THF at 20 mg/mL and added on 20 ml of MilliQ water.

FIG. 17: SEM images of the KL nanoparticles obtained from KL dissolved in THF at 20 mg/mL and added on 40 ml of MilliQ water.

FIG. 18: DLS analysis of the KL nanoparticles obtained from KL dissolved in THF at various temperatures.

FIG. 19: DLS analysis of the KL nanoparticles obtained from KL dissolved in DMSO at various temperatures.

FIG. 20: DLS analysis of the KL nanoparticles obtained from KL dissolved in THF at various stirring speeds.

FIG. 21: DLS analysis of the KL nanoparticles obtained from KL dissolved in DMSO at various stirring speeds.

FIG. 22: DLS spectrum of KL nanoparticles fabricated using DMSO.

FIG. 23: SEM image of KL nanoparticles fabricated using DMSO.

FIG. 24: Helium Ion microscopy (HIM) image of KL nanoparticles fabricated using DMSO.

FIG. 25: DLS spectrum of KL nanoparticles fabricated using THF.

FIG. 26: SEM image of KL nanoparticles fabricated using THF.

FIG. 27: Transmittance measurements of KL nanoparticles of the present disclosure.

FIG. 28: UV Visible spectrum of KL nanoparticles of the present disclosure.

FIG. 29: Differential Scanning Calorimetry (DSC) curves of KL nanoparticles of the disclosure.

FIG. 30: Thermogravimetric Analysis (TGA) curves of KL nanoparticles of the disclosure.

FIG. 31: AFM studies showing the size and Young’s modulus of raw KL.

FIG. 32: AFM studies showing the size and Young’s modulus of KL nanoparticles generated using THF.

FIG. 33: AFM studies showing the size and Young’s modulus of KL nanoparticles generated using DMSO.

FIG. 34: Correlation between size distribution and Young’s modulus of the KL nanoparticles.

FIG. 35: Black and white photograph of polymer nanocomposites (PNCs) with KL as reinforcing filler.

### DETAILED DESCRIPTION OF THE DISCLOSURE

For the disclosure, the following definitions are given:

The miscibility of a solvent is determined by the partition coefficient, namely the logP or the log Kow. It is the logarithm of the ratio between the concentration of the compound to be dissolved into octanol and water. A positive logP reflects a lipophile character of the solvent while a negative logP reflects a hydrophile character of the solvent.

Solubility parameters, such as Hansen solubility parameters, are also used to determine the miscibility of one solvent into another as well as the solubility of KL in different organic solvents. The Hansen solubility parameters comprise δ_{d}, which is a measure of the energy from dispersion forces between molecules; δₚ, which is a measure of the energy from dipolar intermolecular forces between molecules; and δₕ, which is a measure of the energy from hydrogen bonds between molecules. These three parameters are the three coordinates of the Hansen space. The near two molecules are in this three-dimensional space, the more likely they are to dissolve between each other.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g. 1 to 5 can include 1, 2, 3, 4, 5 when referring to, for example, a number of elements, and can also include 1.5, 2, 2.75 and 3.80, when referring to, for example, measurements). The recitation of endpoints also includes the recited endpoint values themselves (e.g. from 1.0 to 5.0 includes both 1.0 and 5.0). Any numerical range recited herein is intended to include all sub-ranges subsumed therein.

The particular features, structures, characteristics or embodiments may be combined in any suitable manner, as would be apparent to a person skilled in the art from this disclosure, in one or more embodiments.

The present disclosure concerns a method for manufacturing a colloidal dispersion of Kraft lignin (KL) nanoparticles. KL is provided in a first step (a). Then, in a second step (b), an organic solution of said KL is prepared by the dissolution of said KL in a single organic solvent. To form the nanoparticles, the solvent-shifting technique requires the addition of an anti-solvent, namely a solvent with no dissolving power of the KL, to trigger the self-assembly and/or the dispersion and thus the formation of colloidal particles. So, in a third step (c), the solution of step (b) is mixed with an antisolvent being or comprising water. In the present disclosure, the organic solution of KL is added to the antisolvent during the third step (c). The addition of the organic solution in water corresponds to the addition of the organic solution in a medium that quenches the growth of the nanoparticles. This drastic increase in the antisolvent reservoir is, therefore, one of the reasons why it is possible to generate nanoparticles of KL having a small size.

With preference, step (c) of mixing the KL solution into water is performed under an inert atmosphere, for instance under argon and/or nitrogen. This prevents the inclusion of air in the medium and subsequently the formation of foam.

The addition of the KL solution in the organic solvent during step (c) is performed dropwise or rapidly.

When the organic solution of KL is added to water, the formation of the KL nanoparticles is carried out instantaneously.

In a preferred implementation of the method of the present disclosure, steps (b) and (c) are performed in a single reactor, or, in other terms, the manufacture of the colloidal dispersion of KL nanoparticles is a one-pot method. A “one-pot method” stands for a method in which the operations related to the dissolution of KL into one or more organic solvents and to the mixing of the solution with an antisolvent being or comprising water are carried out in the same vessel.

By acting on five different parameters, which are the solvents, the KL concentration, the amount of the antisolvent, the temperature and/or the stirring speed at which the mixing of step (c) is carried out, it is possible to control the size of the KL nanoparticles. There is a synergistic effect with regard to the size of the KL nanoparticles when those five parameters are under control.

The narrow values of the PDI (ranging between 0.05 and 0.20) for the KL nanoparticles with a size ranging between 15 nm and 200 nm is to be highlighted.

Additionally, the method of the present disclosures allows for obtaining homogenous KL nanoparticles which do not coalesce together, nor aggregate together. This allows obtaining KL nanoparticles with a well distinguishable morphology (notably by using SEM or HIM analysis). Also, such KL nanoparticles have a good distribution and a good dispersion, notably when used as reinforcing filler for polymer nanocomposites.

### 1^{st} Parameter: Effect of the Solvent

The first parameter concerns the choice of the organic solvent in which KL lignin must be dissolved before being added to the antisolvent. The size of the nanoparticles and the nuclei formation is completely dependent on the diffusion between the antisolvent, i.e. the water, and the organic solvent. The faster is the diffusion, the smaller is the size of the nuclei. Miscibility of DMSO (log Kow: -1.35 ; δ_{d} = 18.4 MPa^{0.5} ; δₚ = 16.4 MPa^{0.5} ; δₕ = 10.2 MPa^{0.5}) with water is much greater than the THF (log Kow: 0.46 ; δ_{d} = 16.8 MPa^{0.5} ; δₚ = 5.7 MPa^{0.5} ; δₕ = 8.0 MPa^{0.5}). Therefore, the diffusion will be faster in the DMSO system than in the THF system. This leads to the formation of smaller nuclei in the DMSO system than in the THF system at the same initial lignin concentration. Assuming that the initial concentration is the same, the number of smaller nuclei in the DMSO system will be higher than the number of smaller nuclei in the THF system. This behaviour predominantly affects the final sizes of KL nanoparticles.

Moreover, it is advantageous that the organic solvent is dried or anhydrous before it is used to dissolve KL.

### 2^{nd} Parameter: Effect of the KL Concentration

The second parameter relates to the initial concentration of KL in the organic solution. To obtain KL nanoparticles with an average diameter size ranging between 15 nm and 200 nm, as determined by Scanning Electron Microscopy studies, and preferably ranging between 15 nm to 70 nm, the KL concentration in the organic solution can be ranging between 15 mg/mL and 55 mg/mL. Advantageously, the KL concentrations in the organic solution can be ranging between 17 mg/mL and 53 mg/mL, more preferentially between 20 mg/mL and 50 mg/mL.

Increasing the KL concentration results in increasing the KL nanoparticles size.

### 3^{rd} Parameter: Effect of the Amount of Anti-Solvent (MilliQ Water)

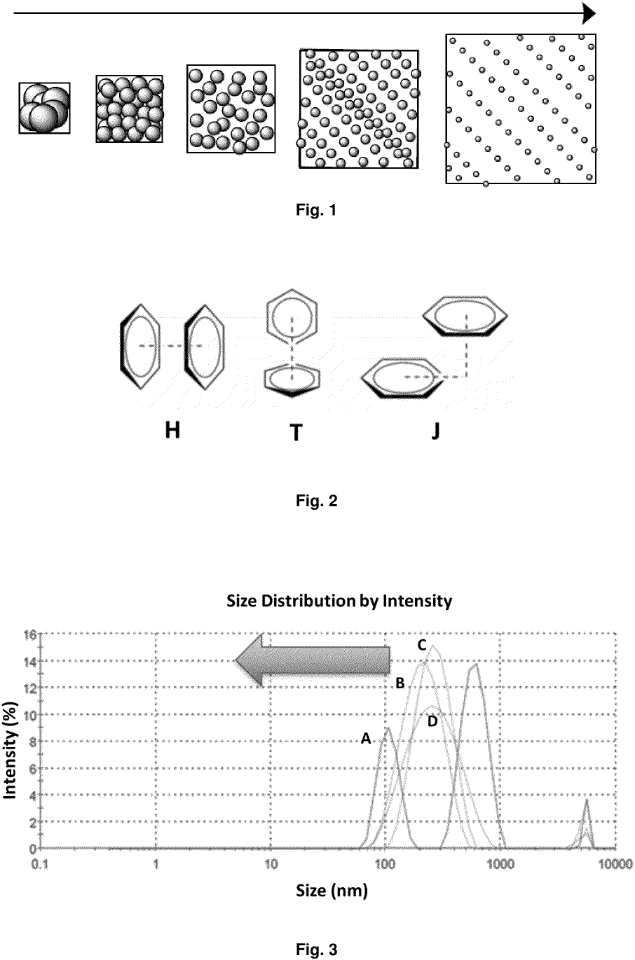

The third parameter concerns the volume of the water in which the organic solution of KL is added. By increasing the amount of antisolvent (i.e. water), it appears that the KL nanoparticles will be more dispersed in the medium, which has for effect to decrease the number of the phenomenon of coalescence and/or Ostwald ripening. FIG. 1 schematically shows that when the water reservoir increases, the nanoparticles have more space between each other, which mean that their growth will be hindered. This effect can be observed in any organic solvents chosen for dissolving KL.

Thus, advantageously, the volume ratio between the antisolvent and the solution of step (b) is ranging between 0.3 and 2.5.

### 4^{th} Parameter: Effect of the Temperature

The fourth parameter relates to the temperature at which the addition of the organic solution of KL onto the water is performed. This parameter is a function of the dissolving power of the organic solvent and the miscibility between the organic solvent and the antisolvent.

When a solvent with a poor dissolving power is used, increasing the temperature is a way to increase the phenomenon of coalescence and/or Ostwald ripening and thus the size of the KL nanoparticles is increasing. For instance, solvents with a poor dissolving power have a partition coefficient superior to -0.50, preferably superior to -0.40, more preferably superior -0.30 and/or a dipole moment inferior to 3 D (< 1.000692285*10⁻²⁹ Cm). Advantageously, such solvents can be 1,4-dioxane, dichloromethane, THF, ethyl acetate and/or acetone, more preferably THF. In this case, the mixing temperature is preferably ranging between 1° C. to 60° C., preferably between 10° C. to 30° C.

However, when a solvent with a good dissolving power is used, the increase of temperature results in increasing the diffusion between the solvent and the water, which leads to more space between the KL nanoparticles in formation and therefore, it helps to obtain KL nanoparticles with a low average diameter size since coalescence and/or Ostwald ripening are avoided. For instance, solvents with high dissolving power have a partition coefficient inferior to -0.50, preferably inferior to -0.60, more preferably inferior to -0.70 and/or are highly polar with a dipole moment superior to 3 D (> 1.000692285 * 10⁻²⁹ Cm). Advantageously, such solvents can be DMSO and/or DMF, more preferably DMSO. In this case, the mixing temperature is preferably ranging between 1° C. and 80° C., more preferably between 10° C. and 70° C.

### 5^{th} Parameter: Effect of the Stirring Speed

The fifth parameter is the stirring speed that is applied during the process of step (c) of mixing the solution of KL into the water. Increasing the stirring speed has for effect to reduce the size of the nanoparticles. With preference, the stirring speed can be ranging between 300 rpm and 2500 rpm, more preferentially between 400 rpm and 2000 rpm. However, at stirring speed above 2500 rpm, preferably above 3000 rpm, more preferably above 3500 rpm, the addition and/or mixing of the KL solution into the antisolvent must be performed under an inert atmosphere (for instance, under argon and/or nitrogen atmosphere) to prevent the formation of foam. Indeed, foaming is caused by the combining effect of the inherent amphiphilic nature of the KL, entrapped air and higher stirring speed. Foaming can be detrimental to the final yield of KL nanoparticles that are obtained.

Advantageously, the one or more organic solvents are removed after the formation of the KL nanoparticles. To completely yield dried KL nanoparticles, a time that is ranging between 3 and 10 days is needed to remove the solvents. Such time is relatively long because it is needed to remove the organic solvent and the antisolvent without de-structuring the KL nanoparticles.

Solvents presenting high boiling points, such as DMSO (b.p.= 189° C.), DMF (b.p. 153° C.) or 1,4-dioxane (b.p. 101° C.), can be removed from the KL nanoparticles using a dialysis process.

Solvents presenting lower boiling points, such as acetonitrile (b.p = 82° C.), dichloromethane (b.p. = 40° C.), tetrahydrofuran (b.p. = 66° C.), ethyl acetate (b.p. = 77° C.) or acetone (b.p. = 56° C.) can be removed from the KL nanoparticles using rotary evaporator.

After complete removal of the solvents, a freeze-drying step can be undertaken to remove the antisolvents, i.e. water, from the KL nanoparticles. Preferentially, the freeze-drying step can be carried out at a temperature ranging between -50° C. and -100° C., more preferentially between -60° C. and -90° C., even more preferentially at -80° C. and/or during a time of at least 24 hours, preferentially of at least 3 days. The step of freeze-drying can also be advantageously carried out at a pressure ranging between 0.05 Pa and 0.20 Pa, more preferentially at a pressure ranging between 0.07 Pa and 0.15 Pa, even more preferentially at 0.12 Pa.

### Characterization and Properties of KL Nanoparticles of the Disclosure

The average diameter size of the nanoparticles is ranging from 9 nm to 200 nm, or from 9 nm to 70 nm, or from 15 nm to 70 nm, as determined by imaging techniques, such as Scanning Electron Microscopy and/or Helium Ion Microscopy.

For example, the average diameter size, as determined by Scanning Electron Microscopy is at least 15 nm, and at most 200 nm, preferably at most 150 nm, more preferably at most 100 nm, even more preferably at most 65 nm, most preferably at most 60 nm, even most preferably at most 50 nm.

For example, the average diameter size, as determined by Scanning Electron Microscopy is at least 20 nm, and at most 200 nm, preferably at most 150 nm, more preferably at most 100 nm, even more preferably at most 65 nm, most preferably at most 60 nm, even most preferably at most 50 nm.

For example, the average diameter size, as determined by Scanning Electron Microscopy is at least 25 nm, and at most 200 nm, preferably at most 150 nm, more preferably at most 100 nm, even more preferably at most 65 nm, most preferably at most 60 nm, even most preferably at most 50 nm

When a solvent with high dissolving power is used to dissolve KL, the average diameter size of the KL nanoparticles obtained with the process according to the disclosure is ranging between 15 nm and 45 nm, preferably between 20 nm and 40 nm.

When a solvent with poor dissolving power is used to dissolve KL, the average diameter size of the KL nanoparticles obtained with the process according to the disclosure is ranging between 40 nm and 90 nm, preferably between 50 nm and 80 nm.

The KL nanoparticles can be re-dispersible in water.

With preference, the nanoparticle having an average diameter size ranging from 15 nm up to 60 nm has a transmittance taken at a wavelength of 600 nm ranging between 40% and 80% as determined by absorption analysis, preferably between 45% and 75%.

The KL nanoparticles have a fluffy aspect. For collecting them, it is preferable to use an electrostatic-free sample collector, because of the formation of high static charges on the nanoparticles.

The three-dimensional structure of the KL nanoparticle comprises at least two types of π-π stacking, as determined by UV-Visible analysis. FIG. 2 is a scheme indicating the three types of π-π stacking in organic compounds comprising aromatic structures, namely the H-shape corresponding to a sandwich-like structure, the T-shaped structure and the J-shaped structure corresponding to a parallel-displaced structure.

The KL nanoparticles have a glass transition temperature (T_{g}) of at least 150° C. as determined by Differential Scanning Calorimetry. With preference, the KL nanoparticles have an additional T_{g} that is ranging between 110° C. and 130° C.

### Use of the KL Nanoparticles as Reinforcing Filler for Polymer Nanocomposites

Polymer nanocomposites (PNCs) are polymers having nanoparticles or nanofiller dispersed into the polymer matrix.

The disclosure provides the use of Kraft lignin nanoparticles as reinforcing filler material in polymer nanocomposites. Examples of PNCs are rubber, plastics or blends of rubber and plastics.

It is preferable that the integration of KL nanoparticles into PNCs be performed by mixing the ingredients using polymer solution casting.

In an example, in the manufacture of PNCs, one or more of the following additional components can be mixed:
at least one base, preferably polydimethylsiloxane (PDMS); and/or
at least one solvent, preferably cyclohexane and/or toluene; and/or
at least one cross-linking agent, preferably siloxane; and/or
KL nanoparticles as reinforcing filler.

The polymer solution casting of all the components mentioned above has led to the formation of a polymer nanocomposite which presents a good dispersion of the filler materials in it. Compared to a polymer nanocomposite devoid of KL nanoparticles, better mechanical properties are obtained-.

With preference, the mixing is carried out by magnetic stirring.

With preference, the mixing is performed at a rotating speed ranging between 800 rpm and 1200 rpm, more preferably between 900 rpm and 1100 rpm.

The mixing can be advantageously performed at a temperature ranging between 15° C. and 110° C., more preferably between 20° C. and 100° C., even more preferably between 25° C. and 90° C., most preferably between 30° C. and 85° C.

The mixing can be advantageously performed for at least 10 minutes, preferably for at least 20 minutes, more preferably for at least 30 minutes, even more preferably for at least 60 minutes, most preferably for at least 90 minutes.

### Use of the KL Nanoparticles in a Cosmetic Composition

The KL nanoparticles of the disclosure can be used in a cosmetic composition, for instance as a delivery carrier of one or more active ingredients, such as a sunblock agent and/or molecules unstable towards oxidations. For instance, the KL nanoparticles can be used as a carrier of ascorbic acid and/or retinoic acid. When used in a cosmetic composition, the administration is advantageously performed topically (i.e., by application on the skin).

### Use of the KL Nanoparticles in a Pharmaceutical Composition

The KL nanoparticles of the disclosure can also be used in a pharmaceutical composition as a drug carrier. The administration of the pharmaceutical composition can be topical or oral.

The cosmetic and pharmaceutical use of the KL nanoparticles is possibly due to the stability of the KL nanoparticles of the disclosure at neutral pH, preferably at a pH ranging between 6 and 9. In acidic pH (e.g. at pH = 2), there will be an increase of hydrogen bonding between the polymer composing the KL nanoparticles, which will have the effect that the structure of the KL nanoparticles induces the release of loaded active ingredients. This is why it is possible to use the KL nanoparticles as a delivery carrier for drugs, chemical compounds, medicines...

### Test and Determination Methods

### Atomic Force Microscope (AFM)

The topography and nanomechanical properties of the samples were thus investigated using an MFP3D Infinity AFM (Asylum Research/Oxford Instruments, Santa Barbara, CA) working in a bimodal AM-FM (Amplitude Modulation-Frequency Modulation) configuration. The samples were prepared by drop-casting the raw lignin, KLNP1 (DMSO system), and KLNP2 (THF system) (100x diluted and dispersed in water) on a silicon wafer and drying it overnight under a fume hood.

In the bimodal AM-FM mode, the nanoscale tip attached to the cantilever is simultaneously excited with two eigenmodes (two different oscillation motions of the cantilever). As the tip approaches the sample surface, the oscillation of the tip is reduced by its interaction. A feedback loop acting on the piezo scanner (Z direction) keeps the amplitude (A₁ ₛₑₜ (112 nm) of the 1^{st} eigenmode (c. 265 KHz) of the cantilever constant to obtain the topography of the sample. The amplitude A_{1,free} away from the surface, was set at 160 nm. The 2^{nd} eigenmode (1.52 MHz) was simultaneously driven at a smaller amplitude A₂ (500 pm) and assist in detecting the frequency shift (Δf₂) via a 90° Phase Lock Loop (φ₁, PLL). The frequency feedback loop maintains the 2^{nd} eigenmode on resonance by a frequency shift (Δf₂) and this is caused due to the change in nanoparticle stiffness. Before calibration, the tip was scanned in contact mode over a TiO₂ surface to round up its apex. The increased tip curvature radius ensured a more stable operation in Elastic Modulus (EM) characterization.

### Dynamic Light Scattering (DLS)

Hydrodynamic particle size and distribution (by determining the polydispersity index PDI) of the KL nanoparticles were measured using a Malvern Zetasizer Nano-ZS90 instrument (UK).

Before analysis, samples were diluted 100 times in water. The refractive index of polystyrene (1.58654 at 632.8 nm) was used as an internal standard value. Measurements were done with a glass cuvette at 25° C. To confirm the reproducibility, three measurements were carried out in each sample. After each analysis, the glass cuvette was washed with MillliQ water and dried using argon. As a hydration layer is formed around the sample during the measurements of the size, the size obtained by DLS is bigger than the size obtained by using the scanning electron microscope.

### Scanning Electron Microscopy (SEM)

SEM images were obtained using Focus Ion Beam (FIB) scanning electronic microscope (model: Helios Nanolab 650), operating at a voltage of 2-30 Kv and current of 13 to 100 pA. Before the SEM analysis, the samples were dried overnight in the open air. Measurements were done in both feel free mode and immersion mode. To confirm the exact size of KL nanoparticles, SEM analyses are done without any metal coating. SEM images were analysed using ImageJ software.

### Helium Ion Microscopy (HIM)

HIM images were obtained using Helium Ion Microscope (HIM: ZEISS ORION NanoFab) from Carl Zeiss Microscopy GmbH. All the Kraft lignin dispersions in DMSO were diluted 100x to visualize a primary particle effectively. The samples were prepared by drop-casting 0.02 mL of lignin dilution onto a silicon wafer and allowing it to dry overnight under a fume hood. The samples were characterized without any conductive coatings. The size and polydispersity of Kraft Lignin nanoparticles were analysed using ImageJ (Version 1.52) and MountainsSPIP 8 software.

As the nanoparticles fabricated using THF are bigger than the nanoparticles fabricated using DMSO, SEM analysis was sufficient to visualize the bigger KL nanoparticles, while it was necessary to perform HIM analysis to visualize the smaller KL nanoparticles. From these two imaging techniques, the shape and the size of the KL nanoparticles were determined.

### Ultra-Violet (UV)-Visible Analysis

π-π stacking of lignin nanoparticles was confirmed with the help of UV-Visible spectroscopy. The multifunctional monochromator-based microplate reader, Tecan infinite M1000Pro, has been used to determine the UV-Visible spectrum. To perform the analysis, dried KL nanoparticles were re-dispersed in MilliQ water (0.025 mg/mL). Samples were placed in the Greiner 96 Flat Bottom Transparent Polystyrol plate. Absorbances were measured from 230 nm to 800 nm wavelengths. 286 scans and 25 flashes were used at 25° C. for each measurement.

### Absorption Analysis

PerkinElmer (LAMBDA 1050+ UV/Vis/NIR) spectrophotometers were used to measure the % transmittance of Lignin Nanodispersion. 3 mL of 20 mg/mL (initial lignin concentration) of each nanodispersion and the deionized water were placed in an acrylic cuvette before the measurement. Double beam arrangements were used to perform the measurement. The percentage of transmittances were noted at 600 nm of wavelength.

### Differential Scanning Calorimetry (DSC)

Glass transition temperature (T_{g}) of the lignin samples was determined using a DSC instrument (DSC 3+, METTLER TOLEDO GmbH) under a nitrogen atmosphere. Before analysis, KL was dried overnight under vacuum at 60° C. During each measurement, approximately 10 mg of dry lignin was used. The samples were heated from room temperature to 120° C. at a heating rate of 10° C./min (first measurement cycle), isothermal for 5 minutes, cooled to 0° C. at a cooling rate of 10° C./min, isothermal for 5 minutes, then reheated to 200° C. at a heating rate of 10° C./min (second measurement cycle). T_{g} was measured from the second measurement cycle.

### Thermogravimetric Analysis (TGA)

Thermal stability and the purity of the lignin samples were determined using a NETZSCH TGA instrument. Before analysis, the lignin samples were dried overnight under vacuum at 60° C. During each measurement, approximately 10 mg of dry lignin was used. The samples were heated from room temperature up to 800° C. at a heating rate of 10° C./min under a nitrogen atmosphere (flow rate = 90 mL min⁻¹).

### EXAMPLES

The embodiments of the present disclosure will be better understood by looking at the example below.

KL has been supplied by UPM BioPiva under the form of BioPiva.

HPLC grade tetrahydrofuran (THF) and anhydrous dimethyl sulfoxide (DMSO) were purchased from Sigma Aldrich.

MilliQ water (0.2 µm PES high flux capsule filter; 18.2 M′Ω.cm at 23° C.) was used as it is from the laboratory.

### General Protocol for Preparing KL Nanoparticles According to the Disclosure

KL (BioPiva) was dissolved in a solvent (1^{st} parameter) to obtain a solution with a certain concentration (2^{nd} parameter). The mixture was stirred at room temperature (25° C.) until a clear solution was obtained. Then, the solution was added into a 1 L of cooled water reservoir with a certain amount of MilliQ water in it (3^{rd} parameter). The solution is added to the water reservoir kept at a certain temperature (4^{th} parameter) under stirring at a certain stirring speed (5^{th} parameter). A glass syringe (50 mL) with a sharp needle (1.00*60 mm) was utilized for the addition of lignin solution into the water reservoir. The mixture was kept stirring for 1 minute. The KL nanoparticles formation took place immediately after the complete addition of KL solution. Particle size was characterized using dynamic light scattering (DLS). Then the organic solvent was removed using either rotary evaporator or dialysis, depending on the boiling point of the organic solvent. After that, water dispersed KL nanoparticles were frozen at -80° C. for overnight using a freezer. Finally, the frozen LNPs were freeze-dried at 0.001 mbar and -110° C. for 3 days using freeze-drier (Christ: Alpha 3-4 LSC basic) to remove the water. Fluffy dried powder samples were stored in glass vials. The five parameters are detailed below.

### 1^{st} Parameter: Effect of the Solvent

Tetrahydrofuran, dimethyl sulfoxide, acetone and 1,-4 dioxane were chosen as a solvent to dissolve lignin at a concentration of 20 mg/mL.

The stirring speed (1000 rpm), the amount of anti-solvent (MilliQ water: 20 mL), the temperature (25° C.) and the addition rate of the KL solution were kept constant.

KL nanoparticles were completed after the immediate addition of lignin solution. Samples were collected and DLS measurement was performed.

FIG. 3 indicates the DLS analyses of the KL nanoparticles obtained when different solvents were used to dissolve the KL. The profile of the KL nanoparticles is shown in function of DMSO (curve A), THF (curve B), acetone (curve C) or 1,4-dioxane (curve D). Uses of DMSO and THF provide KL nanoparticles smaller in size. FIG. 3 is thus a good indication that the effect of polarity and solubility affects the size of the KL nanoparticles.

### 2^{nd} Parameter: Effect of the KL Concentration

Biopiva KL has been dissolved in THF at the following concentrations: 20 mg/mL, 30 mg/mL, 40 mg/mL, 50 mg/mL and 60 mg/mL (experiments I to V). For experiment VI, Biopiva KL has been dissolved in DMSO at a concentration of 20 mg/mL.

The stirring speed (1000 rpm), the amount of anti-solvent (MilliQ water: 20 mL), the temperature (25° C.) and the addition rate of the KL solution were kept constant.

KL nanoparticles were completed after the immediate addition of lignin solution. Samples were collected and DLS measurement was performed.

Table 1 indicates the results of the nanoparticles formations.

TABLE 1

Results of nanoparticles formation carried out by varying the KL concentration

| | I | II | III | IV | V | VI | VII | VIII | IX | X |
|---|---|---|---|---|---|---|---|---|---|---|
| Solvent | THF | | | | | DMSO | | | | |
| Concentrations (mg/mL) | 20 | 30 | 40 | 50 | 60 | 20 | 30 | 40 | 50 | 60 |
| Size (nm) | 100 | 125 | 155 | 185 | 235 | 70 | 90 | 125 | 175 | 230 |
| PDI | 0.12 | 0.13 | 0.12 | 0.12 | 0.38 | 0.18 | 0.18 | 0.23 | 0.30 | 0.40 |

The narrow values of the polydispersity index (PDI), ranging between 0.12 and 0.30 for nanoparticles of a size ranging between 70 nm and 185 nm is here highlighted. This means that the majority of the KL nanoparticles synthesized by this method are within the said range concerning their size.

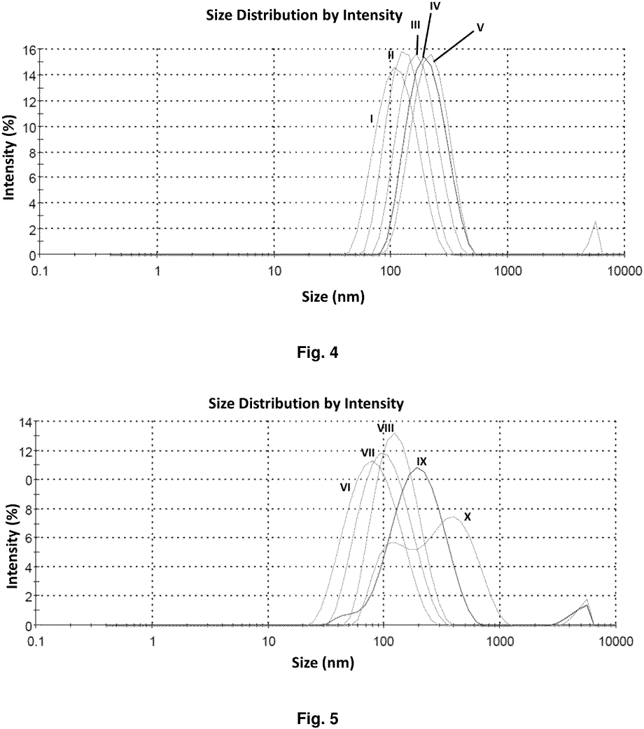

FIGS. 4 and 5 respectively show the DLS analysis of KL nanoparticles obtained with experiments I to V (in THF) and VI to X (in DMSO). It is therefore evident in light of these experiments that by increasing the concentration of KL into the dissolving solvent, the size of the KL nanoparticles increases.

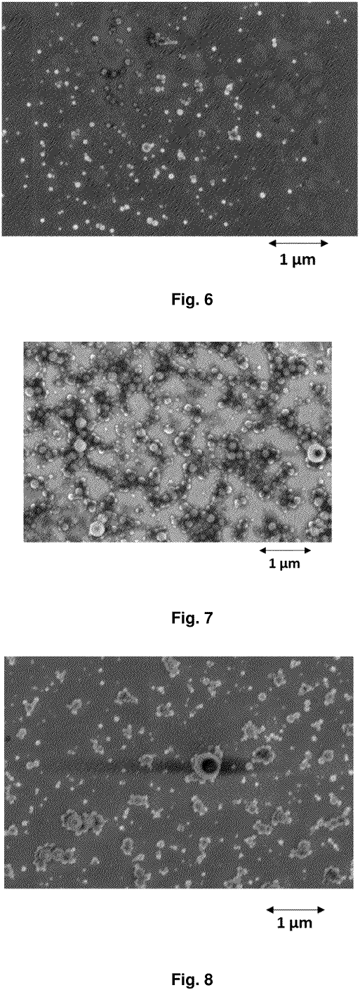

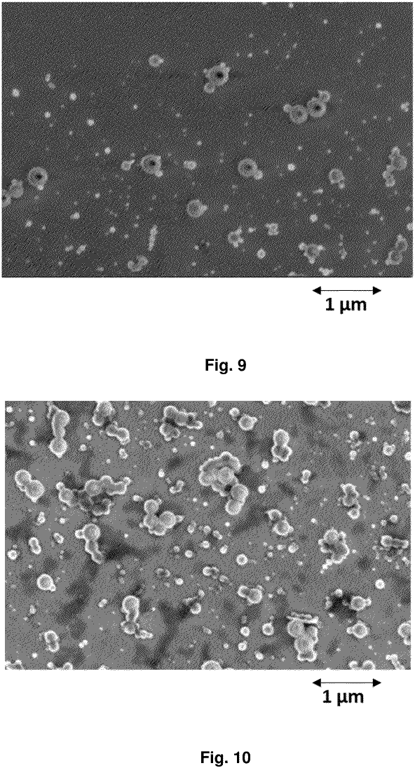

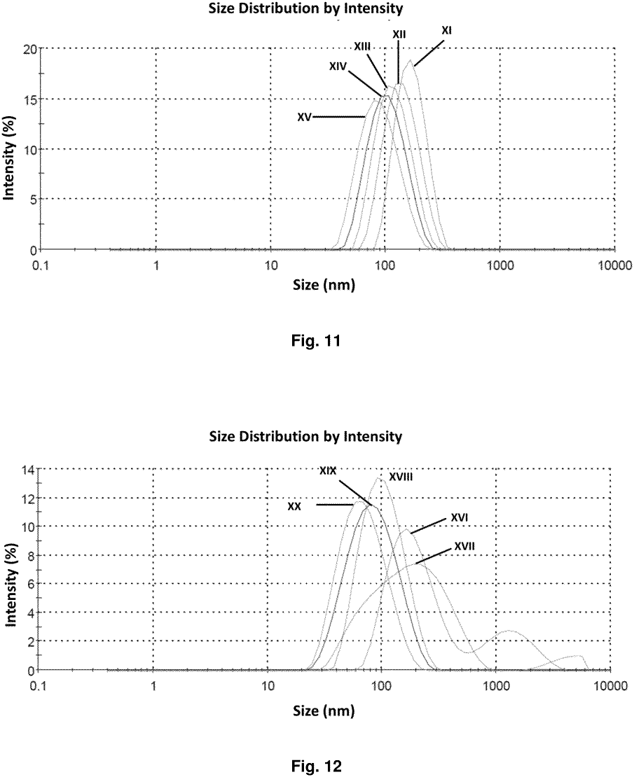

SEM images of the KL nanoparticles obtained with experiments I to V are respectively displayed in FIGS. 6 to 10.

### 3^{rd} Parameter: Effect of the Amount of Anti-Solvent (MilliQ Water)

Experiments VII to XI in THF and XII to XV in DMSO have been carried out to determine the effect of the amount of the antisolvent, namely water, on the size and distribution of the KL nanoparticles.

The amounts of 4 mL, 8 mL, 12 mL, 20 mL and 40 mL were chosen as the amount of water.

The KL concentration (20 mg/mL), choice of solvent (THF or DMSO), the stirring speed (1000 rpm), the temperature (25° C.) and the addition rate of the KL solution were kept constant.

KL nanoparticles were completed after the immediate addition of lignin solution. Samples were collected and DLS measurement was performed.

Table 2 indicates the results of the nanoparticles formations.

TABLE 2

Results of nanoparticles formation carried out by varying the amount of antisolvent

| | XI | XII | XIII | XIV | XV | XVI | XVII | XVIII | XIX | XX |
|---|---|---|---|---|---|---|---|---|---|---|
| Solvent | THF | | | | | DMSO | | | | |
| Amount of MilliQ water (mL) | 4 | 8 | 12 | 20 | 40 | 4 | 8 | 12 | 20 | 40 |
| Ratio water / KL solution | 0.2 | 0.4 | 0.6 | 1 | 2 | 0.2 | 0.4 | 0.6 | 1 | 2 |
| Size (nm) | 160 | 130 | 110 | 95 | 80 | 205 | 135 | 95 | 75 | 65 |
| PDI | 0.10 | 0.10 | 0.12 | 0.11 | 0.13 | 0.35 | 0.28 | 0.16 | 0.18 | 0.20 |

It can be seen that increasing the ratio between the water and the KL solution favours the formation of smaller KL nanoparticles. These findings are observed either in THF or in DMSO.

FIG. 11 displays the DLS curve for experiments XI, to XV, while FIG. 12 displays the DLS curve for experiments XVI to XX.

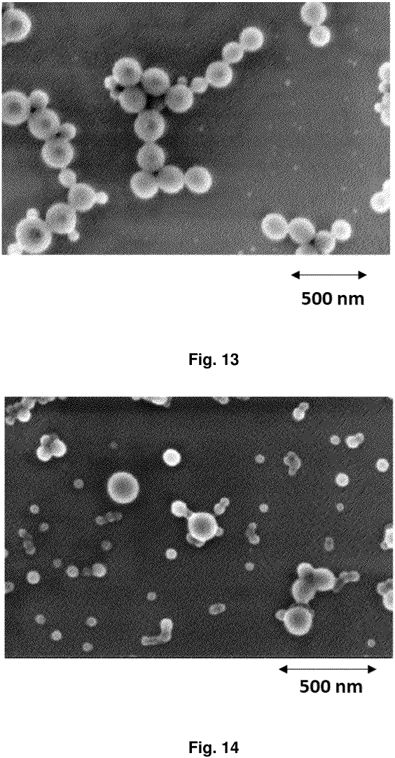

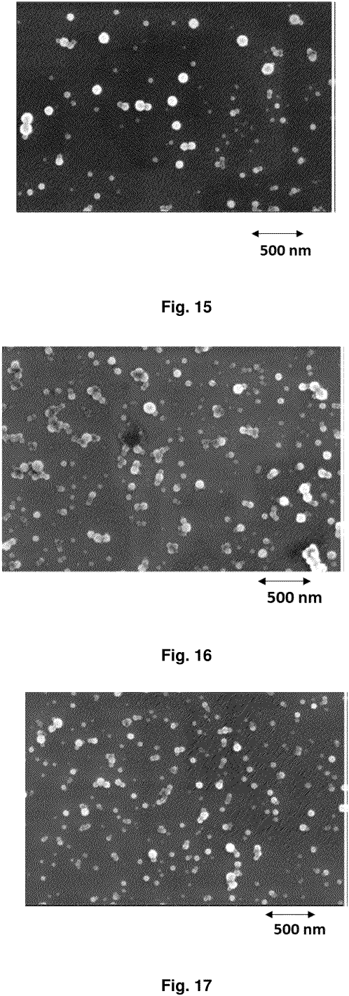

SEM images of the KL nanoparticles obtained with experiments Xl to XV are respectively displayed in FIGS. 13 to 17.

### 4^{th} Parameter: Effect of the Temperature

The temperature of the water bath, before the addition of the KL solution, can be heated. Experiments XXI to XXV concern a KL solution in THF, and subsequently, the tested temperatures were 1° C., 20° C., 40° C., 60° C. and 80° C. Experiments XXVI to XXX concern a KL solution in DMSO. The tested temperatures were the same as for THF.

The KL concentration (20 mg/mL), choice of solvent (THF or DMSO), the stirring speed (1000 rpm), and the addition rate of the KL solution were kept constant.

KL nanoparticles were completed after the immediate addition of lignin solution. Samples were collected and DLS measurement was performed.

Table 3 indicates the results of the nanoparticles formations.

TABLE 3

Results of nanoparticles formation carried out by varying the temperature of the water bath

| | XXI | XXII | XXXIII | XXIV | XXV |
|---|---|---|---|---|---|
| Solvent | THF | | | | |
| Temperature (°C) | 1 | 20 | 40 | 60 | 80 |
| Size (nm) | 95 | 110 | 130 | 195 | 280 |
| PDI | 0.09 | 0.10 | 0.14 | 0.16 | 0.36 |

TABLE 3 - continued

| | XXVI | XXVII | XXVIII | XXIX | XXX |
|---|---|---|---|---|---|
| Solvent | DMSO | | | | |
| Temperature (°C) | 1 | 20 | 40 | 60 | 80 |
| Size (nm) | 90 | 70 | 60 | 54 | 60 |
| PDI | 0.2 | 0.16 | 0.14 | 0.12 | 0.10 |

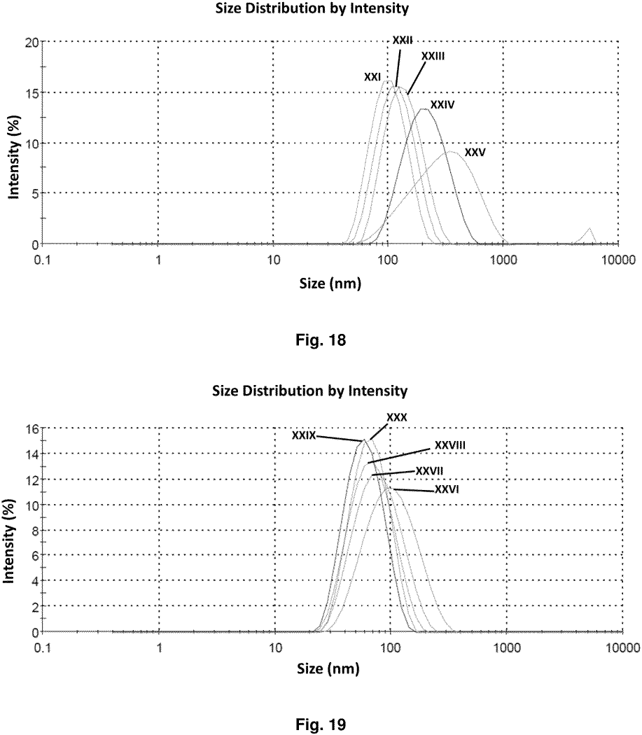

These results, also displayed in FIG. 18, show that in THF, the higher is the temperature of the water bath, the bigger is the size of the obtained KL nanoparticles. As displayed in FIG. 19, the trend is opposite when DMSO is used as the dissolving solvents of the KL. This is due to the better dissolving power of DMSO than the dissolving power of THF.

The fact that KL dissolves better in DMSO than in THF allows for the formation of smaller nuclei that are free to move into the medium. By adding the KL solution to the antisolvent (i.e., water), the small nuclei will form smaller nanoparticles. In THF, as the dissolution of the KL is less good, the nuclei will be bigger. By increasing the temperature, the phenomenon of coalescence and/or Ostwald ripening will increase, leading to bigger KL nanoparticles than if the process was conducted in the presence of a better solvent.

### 5^{th} Parameter: Effect of the Stirring Speed

The stirring speed for the KL nanoparticles has also been experimented.

The KL concentration (20 mg/mL), choice of solvent (THF or DMSO), the temperature (25° C.), and the addition rate of the KL solution were kept constant.

KL nanoparticles were completed after the immediate addition of lignin solution. Samples were collected and DLS measurement was performed.

Table 4 indicates the results of the nanoparticles formations.

TABLE 4

Results of nanoparticles formation carried out by varying the stirring speed in the water bath

| | XXXI | XXXII | XXXIII | XXXIV | XXXV |
|---|---|---|---|---|---|
| Solvent | THF | | | | |
| Stirring speed (rpm) | 400 | 800 | 1200 | 1600 | 2000 |
| Size (nm) | 135 | 120 | 110 | 105 | 100 |
| PDI | 0.10 | 0.10 | 0.12 | 0.13 | 0.12 |

TABLE 4 - continued

| | XXXVI | XXXVII | XXXVIII | XXXIV | XL |
|---|---|---|---|---|---|
| Solvent | DMSO | | | | |
| Stirring speed (rpm) | 400 | 800 | 1200 | 1600 | 2000 |
| Size (nm) | 145 | 80 | 73 | 73 | 70 |
| PDI | 0.28 | 0.18 | 0.16 | 0.20 | 0.20 |

It has been observed that at elevated stirring speed (superior to 2000 rpm), foam started to form.

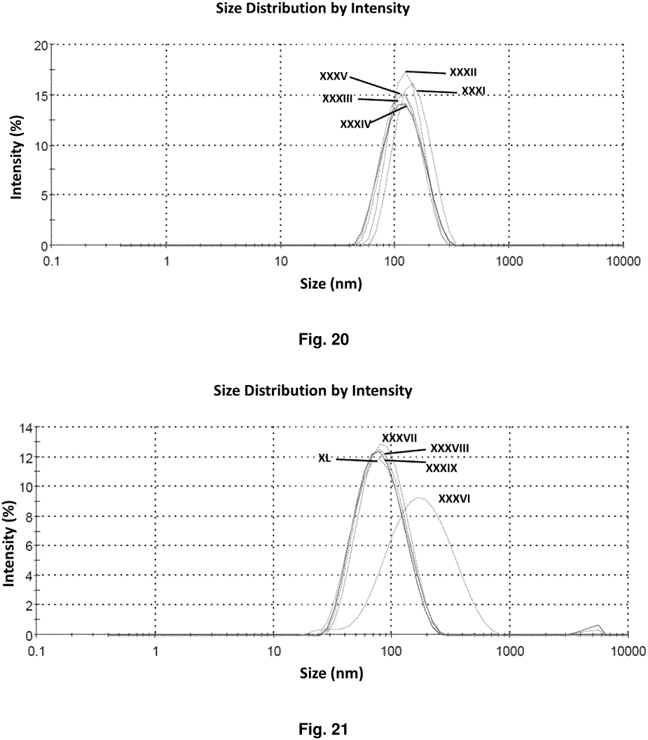

The DLS analysis for the stirring speed are shown respectively in FIGS. 20 and 21 and it is clear that upon an increase of the stirring speed, the size of the KL nanoparticles decreases.

### Synthesis of KL Nanoparticles With a Diameter Size Ranging Between 9 Nm and 45 Nm

1 g of KL (BioPiva) was dissolved in 50 mL of DMSO. The mixture was stirred at room temperature (25° C.) until a clear solution was obtained. Then the solution was added into a 1 L water reservoir, which was stirring at a speed of 1000 rpm at room temperature (25° C.). The temperature of the water reservoir was kept at 25° C. A glass syringe (50 mL) with a sharp needle (1.00*60 mm) was utilized for the addition of lignin solution into the water reservoir. The mixture was kept stirring for 1 minute. The KL nanoparticles formation took place immediately after the complete addition of KL solution. Then DMSO was removed using dialysis at room temperature for 4 days. After that, water dispersed were frozen at -80° C. for overnight using a freezer. Finally, the frozen KL nanoparticles were freeze-dried at 0.001 mbar and -110° C. for 4 days using freeze-drier (Christ: Alpha 3-4 LSC basic). Fluffy dried powder samples were stored in glass vials. The final yield of the sample was 90%.

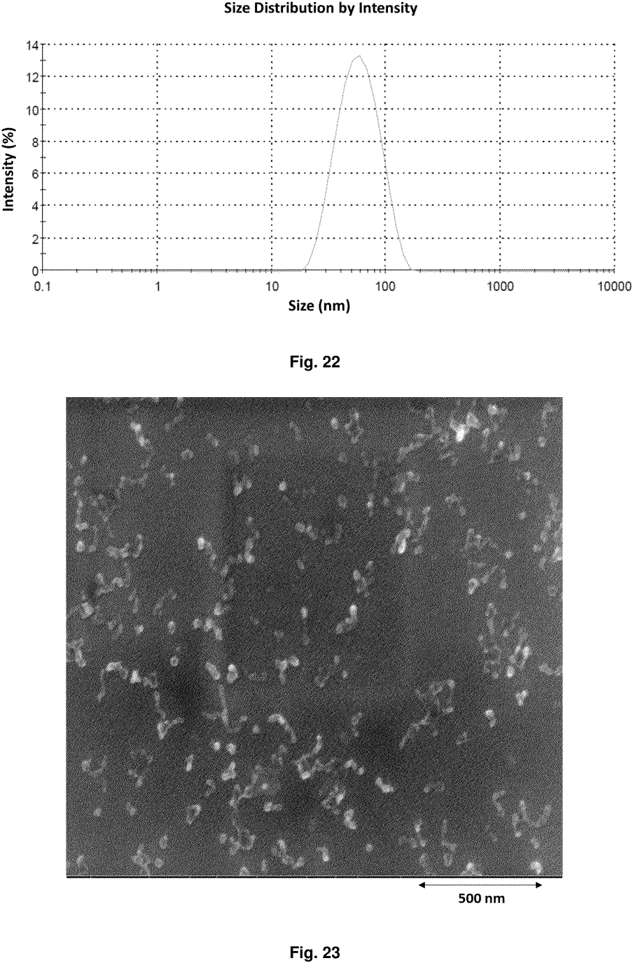

The DLS analysis, indicated in FIG. 22, confirms that the hydrodynamic radius of the KL nanoparticles is 55 nm with a narrow polydispersity index of 0.18.

The SEM analysis, shown in FIG. 23, confirmed that the average diameter size of the dried KL nanoparticles is 15 nm. Most of the nanoparticles, namely between 80% and 90% of the nanoparticles, present an average diameter size which is 15 nm, also confirming the narrow polydispersity index of 0.18.

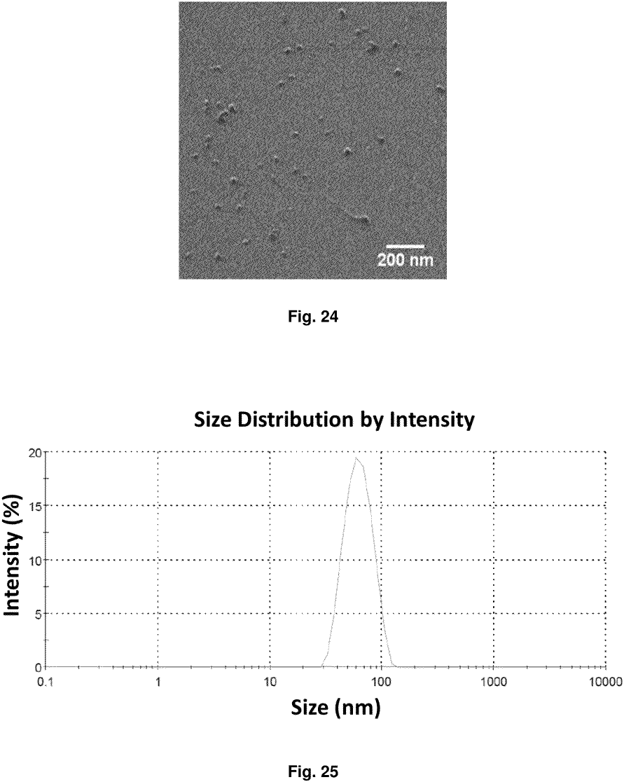

The HIM analysis, shown in FIG. 24, also confirmed that the average diameter size of the dried KL nanoparticles is about 15 nm (more specifically 17 nm, with a standard deviation of 8 nm). HIM analysis is therefore a confirmation that KL nanoparticles having an average diameter size as low as 9 nm or 10 nm can be obtained. The HIM analysis further confirms the sphericity of the KL nanoparticles fabricated using the DMSO system.

### Synthesis of KL Nanoparticles With a Diameter Size Ranging Between 50 Nm and 70 Nm

1 g of KL (BioPiva) was dissolved in 50 mL of THF. The mixture was stirred at room temperature (25° C.) until a clear solution was obtained. Then the solution was added into a 1 L of cooled water reservoir, which was stirring at a speed of 1000 rpm. The temperature of the water reservoir was kept at 10° C. A glass syringe (50 mL) with a sharp needle (1.00*60 mm) was utilized for the addition of lignin solution into the water reservoir. The mixture was kept stirring for 1 minute. The KL nanoparticles formation took place immediately after the complete addition of KL solution. Particle size was characterized using dynamic light scattering (DLS). Then THF was removed using a rotary evaporator. After that, water dispersed KL nanoparticles were frozen at -80° C. for overnight using a freezer. Finally, the frozen LNPs were freeze-dried at 0.001 mbar and -110° C. for 3 days using freeze-drier (Christ: Alpha 3-4 LSC basic). Fluffy dried powder samples were stored in glass vials. The final yield of the sample was 80%.

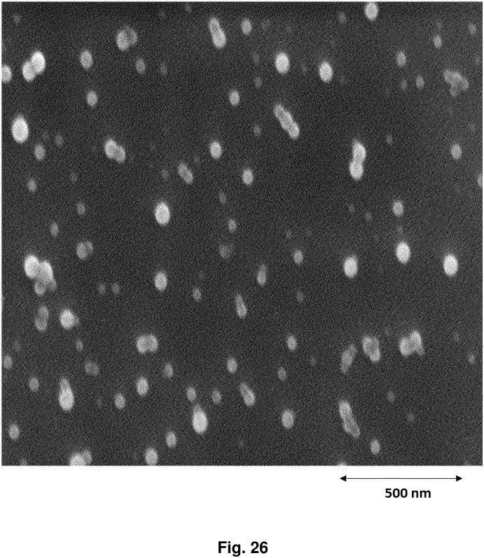

FIG. 25 indicates the DLS spectrum of the KL nanoparticlespresenting a hydrodynamic radius of 80 nm with a narrow PDI of 0.15.

The SEM image is given in FIG. 26 and confirms that the average diameter size of the dried KL nanoparticles is 60 nm. The SEM image further confirms the sphericity of the KL nanoparticles fabricated using the THF system.

### Characterization of the KL Nanoparticles

### Characterization by Absorption Analysis

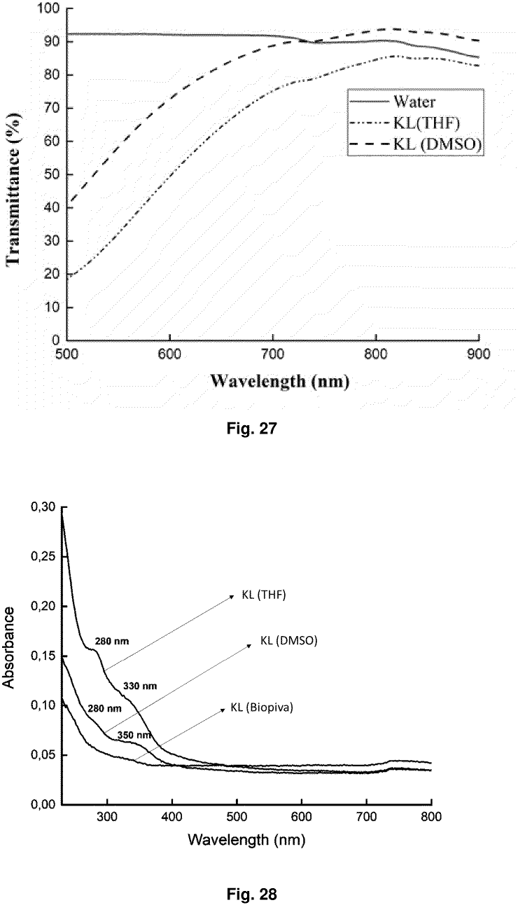

The KL nanoparticles, dispersed in water, which have an average diameter size below 60 nm, do not coalesce together neither form aggregates. FIG. 27 shows the transmittance of incident light having a wavelength ranging between 500 nm and 900 nm measured on the KL nanoparticles. This analysis was performed at an initial lignin concentration of 20 mg/mL. Table 5 indicates the obtained results at 600 nm.

TABLE 5

Transmittance measurements

| Sample | Size^{a} | transmittance at 600 nm^{b} |
|---|---|---|
| water | n.a.^{c} | 92% |
| KL (THF) | 60 nm | 49% |
| KL (DMSO) | 15 nm | 72% |

^{a}as determined by Scanning Electron Microscopy
^{b} as determined by Absorption analysis
^{c} non applicable

### Characterization by UV

As KL is an organic compound comprising aromatic structures, UV-Visible analyses have revealed that there is the presence of two different kinds of π-π stacking in the KL nanofabrication. FIG. 28 shows that the UV visible spectrum of the KL nanoparticles fabricated in THF and DMSO (in comparison with the spectrum of the KL) and the absorbance at 280 nm indicates the presence of J aggregates, namely of parallel-displaced compounds. Such absorbance in lignin nanoparticles has already been reported in the study entitled “Preparation and characterization of lignin nanoparticles: evaluation of their potential as antioxidants and UV protectants”, from Rao Yearla S. et al. (J. Exp. Nanosci., 2016, 11 (4), 289-302).

A second type of π-π stacking has been observed by UV-Visible analysis, at a wavelength ranging between 315 nm and 365 nm, more specifically at 330 nm for the KL nanoparticles fabricated using THF and at 350 nm for the KL nanoparticles fabricated using DMSO. Since the H aggregates reflect the repulsive forces caused by a symmetric cloud of molecules (as schematically shown in the last square of FIG. 1), it is assumed that the absorbance at 350 nm is due to the H aggregates occurring when DMSO is used to dissolve KL. The absorbance at 330 nm is rather due to the T-shaped structure occurring when THF has been employed to fabricate the KL nanoparticles, since it reflects the asymmetricity in the formation of KL nanoparticles, probably due to a lack of diffusion in comparison with the system where DMSO is used.

### Characterization by DSC

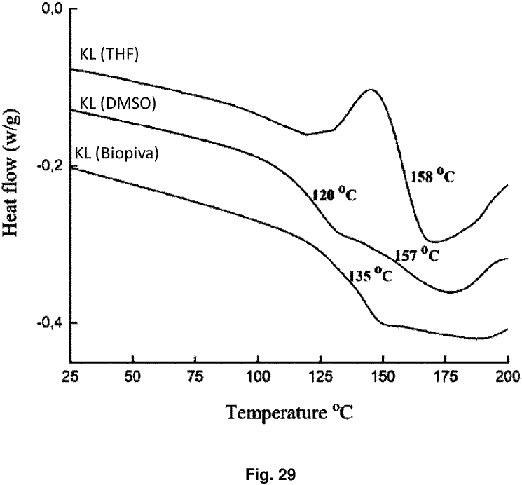

FIG. 29 shows the DSC curve of the KL nanoparticles of the disclosure in comparison with KL and reveals that the KL nanoparticles have a glass transition temperature (T_{g}) of 157° C. and 158° C. for the KL nanoparticles fabricated respectively in DMSO and THF. This T_{g} is higher than the T_{g} for the KL. Such kind of results is thus demonstrated for the first time. As an elevated T_{g} is an indication that more energy is required to break the physical interaction between two components, it is remarkable that the stability of the KL nanoparticles of the disclosure is considerably enhanced.

Moreover, for the KL nanoparticles fabricated in THF, the exothermic hump is the evidence of the energy release while de-structuring the self-assembly of the KL nanoparticles and the hump itself corroborates the assumption that the π-π stacking, determined thanks to the UV-Visible analysis, is of the T-shape.

For the KL nanoparticles fabricated in DMSO, a second T_{g} has been observed at 120° C. The presence of two glass transition temperatures indicates that the KL nanoparticle comprises two segmental arrangements. T_{g} at 120° C. reflects the π-π stacking of the H-type, since this kind of π-π stacking involves repulsive forces and thus demands less energy to break down. This second T_{g} corresponds to the breakdown of the polymer chains that constitute the KL in the KL nanoparticles.

### Stability of the KL Nanoparticles

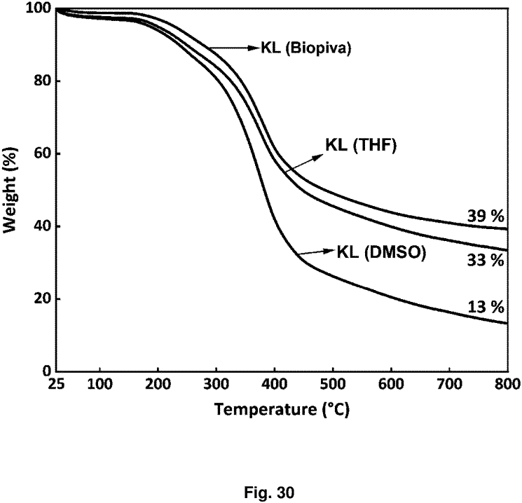

FIG. 30 shows the TGA analysis of the KL nanoparticles of the disclosure in comparison with the KL. As displayed, the degradation of KL starts from 220° C. 39% char yield of KL is observed. The KL nanoparticles fabricated in DMSO and THF have a similar curve, although more stable than KL, with respectively 13% char yield and 33% char yield. % char yield is equivalent to the residual weight % of the material on which the TGA analysis is carried out. The smaller nanoparticles have a higher surface area and high thermal conductivity, which catalyse their degradation.

### Elastic Deformation of the KL Nanoparticles

The bimodal AM-FM analyses allow to simultaneously obtain topography and elastic modulus (Young’s modulus) images.

The bimodal AM-FM mode provides the quantitative mapping of the nanomechanical properties of a surface, as indicated in the study of Kocun M., et al. entitled “Fast, High Resolution, and Wide Modulus Range Nanomechanical Mapping with Bimodal Tapping Mode” (ACS Nano, 2017, 11 (10), 10097-10105).

The cantilever stiffness of the AFM tip (AC160TS, Olympus, Japan), k₁ (28.9 N/m), and k₂ (952 N/m), at its 1^{St} (f₁) and 2^{nd} (f₂) eigenmodes, as well as the inverse optical sensitivity, are calibrated using the Sadler non-contact method before the measurements. The quality factor Q₁ (406) is extracted during the amplitude tune of A₁, _{free} (160 nm) of the 1^{st} eigenmode.

The Hertz model for contact mechanics was applied for the analytical calculation of modulus mechanics (see studies of Benaglia S. et al., entitled “Fast and high-resolution mapping of elastic properties of biomolecules and polymers with bimodal AFM” (Nat. Protoc., 2018, 13 (12), 2890-2907) and of Labuda A. et al., entitled “Generalized Hertz model for bimodal nanomechanical mapping” (Beilstein J. Nanotechnol., 2016, 7, 970-982). The calibrated cantilever parameters (k₁, k₂, f₁, f₂ and Q₁), combined with readings collected during the analysis (A₁, φ₁ and Δf₂), allow the calculation of the indentation depth δ (Eq. 1) of the tip on the surface and the effective storage modulus E_{eff} ( Eq. 2).

In the case of flat punch tip:

$$δ = \frac{3}{4} \frac{{k}_{1}}{{Q}_{1}} {A}_{1 , f r e e} c o s {φ}_{1} \frac{{f}_{2}}{2 {k}_{2} Δ {f}_{2}} \qquad \text{Eq. 1}$$

$${E}_{e f f} = \sqrt{\frac{2}{3}} \frac{π}{R} {\left(\frac{{k}_{1}}{{Q}_{1}} \frac{{A}_{1 , f r e e}}{{A}_{1 , s e t}} c o s {φ}_{1}\right)}^{- \frac{1}{2}} {\left(2 {k}_{2} \frac{Δ {f}_{2}}{{f}_{2}}\right)}^{\frac{3}{2}} \qquad \text{Eq.2}$$

From equation 2, the AFM tip radius (R) is the only free parameter to be determined and modelized as a flat punch. This parameter is determined by analysing a modulus reference sample (polystyrene/polycaprolactone). The EM of the polystyrene phase is 2.7 GPa and then the tip radius was extracted.

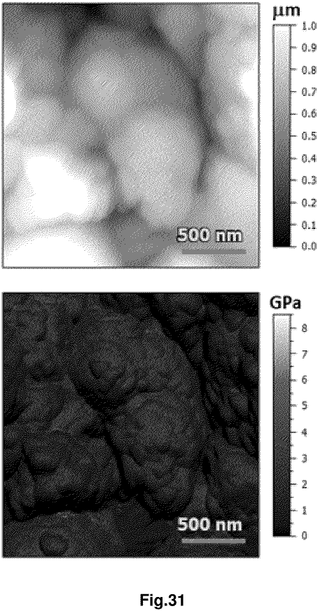

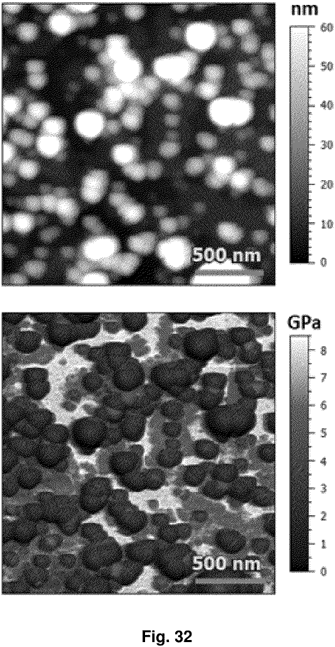

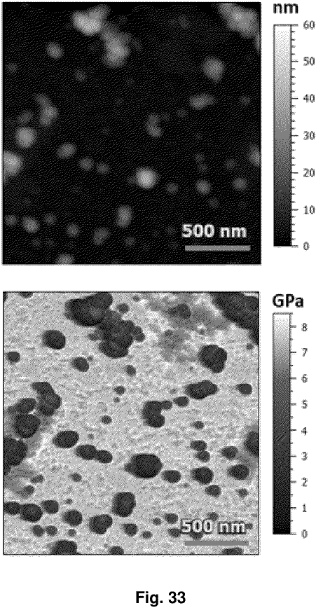

The indentation depth (δ: 800 pm) contributed to measure the sample topography. The effective storage modulus was calculated using the above-measured variables. The nanoparticles are individually segmented by a watershed mode in MountainSPIP 8 software (Digisurf, France). The mask obtained is then applied to the EM image to obtain maximum height (assumed to be the diameter) and the corresponding average EM of the same particle, as shown in FIG. 31 (for the raw KL), FIG. 32 (for the KL nanoparticles generated using the THF system) and FIG. 33 (for the KL nanoparticles generated using the DMSO system).

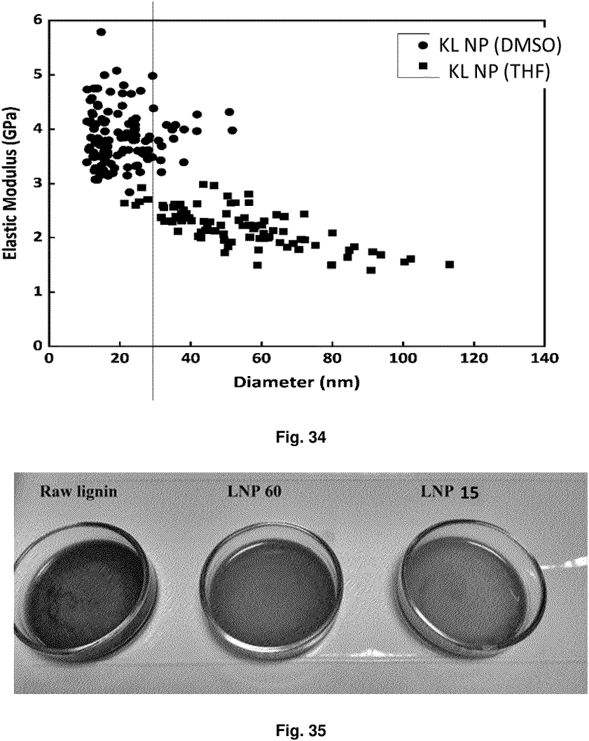

FIG. 34 shows the KL nanoparticles size distribution correlated to Young’s modulus derived from Atomic Force Microscopy experiments.

Therefore, it was determined that said KL nanoparticle has Young’s modulus ranging between 1.0 GPa and 6.0 GPa as determined by Atomic Force Microscopy, preferentially between 1.4 GPa and 5.1 GPa, more preferentially between 1.5 GPa and 5.0 GPa.

For example, the KL nanoparticles generated using the THF system have Young’s modulus ranging between 1.0 GPa and 3.0 GPa as determined by Atomic Force Microscopy, preferentially between 1.4 GPa and 2.9 GPa, more preferentially between 1.5 GPa and 2.8 GPa, even more preferentially between 1.6 GPa and 2.7 GPa.

For example, the KL nanoparticles generated using the DMSO system have Young’s modulus ranging between 2.7 GPa and 6.0 GPa, preferentially between 2.8 GPa and 5.1 GPa, more preferentially between 2.9 GPa and 5.0 GPa, even more preferentially between 3.0 GPa and 4.9 GPa.

### KL Nanoparticles as Reinforcing Material Into Polymer Nanocomposite

In an example, a polymer nanocomposite (PNC) has been formed using the solution casting process.

A mixture of polydimethylsiloxane (PDMS) in cyclohexane was prepared. Kraft lignin (as raw lignin or as nanoparticles with an average diameter size of 15 nm or as nanoparticles with an average diameter size of 60 nm) were then dispersed in said mixture. Magnetic stirring of the mixture was performed at 1000 rpm at room temperature for 30 minutes then the cross-linking agent, i.e. the siloxane, was added into the mixture. The PDMS/siloxane ratio is 10/1. After evaporating the cyclohexane, the resulting viscous solution was poured into a Petri dish. Then the Petri dish was kept under vacuum for 30 minutes to eliminate voids and finally, the samples were cured at 100° C. for 35 minutes.

A control experiment has also been performed without dispersing Kraft lignin.

The PDMS that has been used is a vinyl-terminated PDMS, for example Sylgard® 184, which is available at Sigma-Aldrich (product number 761028). The kinematic viscosity of the product at 25° C. is ranging between 4000.00 cSt and 6500.00 cSt.

The cross-linking agent is a cyclotetrasiloxane, such as octamethyltetrasiloxane.

Table 6 indicates the amounts of the different components in the polymer solution casting experiments.

TABLE 6

Overview of the formation of PNCs without filler (control) and with fillers

| | | | | |
|---|---|---|---|---|
| Components | Control | Raw Lignin | KL nanoparticles 15 nm | KL nanoparticles 60 nm |
| Base: (PDMS) | 2 g | 2 g | 2 g | 2 g |
| Filler: Kraft Lignin | --- | 10 mg | 10 mg | 10 mg |
| Cross-linking agent: (Siloxane) | 0.2 g | 0.2 g | 0.2 g | 0.2 g |
| Solvent: Cyclohexane | 10 mL | 10 mL | 10 mL | 10 mL |

FIG. 35 is a photograph of the Petri dish of the experiments conducted with a filler. The first Petri dish (from left to right) is when using raw lignin, the second Petri dish is when using KL nanoparticles with an average diameter of 15 nm and the third Petri dish is when using KL nanoparticles with an average diameter of 60 nm. From these photographs, it is clear that the incorporation of the KL nanoparticles of the disclosure has a beneficial effect on the structure of the surface of the obtained PNC.

1-41. (canceled)

42. Method for manufacturing a colloidal dispersion of Kraft lignin nanoparticles said method is characterized in that it comprises the following steps:
a) providing Kraft lignin;
b) dissolving said Kraft lignin into a solvent, to obtain a solution having a concentration of Kraft lignin of at least 15 mg/ml;
c) mixing the solution of step (b) with an antisolvent under mixing conditions, to provide a colloidal dispersion of nanoparticles having an average diameter size ranging between 9 nm and below 15 nm as determined by Helium Ion Microscopy and an average diameter size ranging between 15 nm and 70 nm as determined by Scanning Electron Microscopy;
wherein the solvent used in step (b) of dissolving said Kraft lignin is one or more organic solvents, and wherein step (c) of mixing is performed by the addition of the solution of step (b) into an antisolvent being or comprising water.

43. The method according to claim 42, characterized in that said one or more organic solvents have a partition coefficient inferior to -0.50.

44. The method according to claim 43, characterized in that said one or more organic solvents are selected from dimethyl sulfoxide, dimethylformamide and any mixture thereof.

45. The method according to claim 43, characterized in that said solution of step (b) has a concentration of Kraft lignin ranging between 15 mg/ml and 35 mg/ml.

46. The method according to claim 42, characterized in that said one or more organic solvents have a partition coefficient superior to -0.50.

47. The method according to claim 46, characterized in that said one or more organic solvents are selected from 1,4-dioxane, dichloromethane, tetrahydrofuran, ethyl acetate, acetone, and any mixture thereof.

48. The method according to claim 46, characterized in that the solution of step (b) has a concentration of Kraft lignin ranging between 15 mg/ml and 55 mg/ml.

49. The method according to claim 42, characterized in that steps (b) and (c) are performed in a single reactor.

50. Method for manufacturing lignin nanoparticles, said method comprising the method for manufacturing a colloidal dispersion of Kraft lignin nanoparticles according to claim 42, said method for manufacturing lignin nanoparticles being characterized in that step (c) is followed by the step (d) of removing said one or more organic solvents.

51. The method according to claim 50, characterized in that step (d) of removing said one or more organic solvents is performed by evaporation with pressured controlled rotary evaporator.

52. The method according to claim 50, characterized in that step (d) of removing said one or more organic solvents is performed by dialysis.

53. The method according to claim 50, characterized in that said step (d) is followed by a step (e) of freeze-drying.

54. Kraft lignin nanoparticle, said nanoparticle being characterized in that said nanoparticle is spherical as determined by Helium Ion Microscopy and Scanning Electron Microscopy, in that said nanoparticle has an average diameter size ranging between 9 nm and below 15 nm as determined by Helium Ion Microscopy and an average diameter size ranging between 15 nm and 70 nm as determined by Scanning Electron Microscopy, and in that said nanoparticle has a glass transition temperature which is superior to the glass transition temperature of the Kraft lignin as determined by Differential Scanning Calorimetry, wherein the Differential Scanning Calorimetry is conducted (i) by heating the nanoparticle with a first measurement cycle from room temperature to 120° C. at a heating rate of 10° C./min, (ii) by keeping at 120° C. for 5 minutes; (iii) by cooling to 0° C. at a cooling rate of 10° C./min and (iv) by reheating with a second measurement cycle to 200° C. at a heating rate of 10° C./min, the glass transition temperature being measured from the second measurement cycle.

55. The Kraft lignin nanoparticle according to claim 54, characterized in that said nanoparticle has a transmittance taken at a wavelength of 600 nm ranging between 40% and 80% as determined by absorption analysis made in deionized water at a concentration of 20 mg/ml.

56. The Kraft lignin nanoparticle according to claim 54, characterized in that said glass transition temperature which is superior to the glass transition temperature of the Kraft lignin as determined by Differential Scanning Calorimetry is of at least 150° C., wherein the Differential Scanning Calorimetry is conducted (i) by heating the nanoparticle with a first measurement cycle from room temperature to 120° C. at a heating rate of 10° C./min, (ii) by keeping at 120° C. for 5 minutes; (iii) by cooling to 0° C. at a cooling rate of 10° C./min and (iv) by reheating with a second measurement cycle to 200° C. at a heating rate of 10° C./min, the glass transition temperature being measured from the second measurement cycle.

57. The Kraft lignin nanoparticle according to claim 54, characterized in that said glass transition temperature which is superior to the glass transition temperature of the Kraft lignin as determined by Differential Scanning Calorimetry is a second glass transition temperature and in that said nanoparticles has a first glass transition temperature as determined by Differential Scanning Calorimetry, wherein the Differential Scanning Calorimetry is conducted (i) by heating the nanoparticle with a first measurement cycle from room temperature to 120° C. at a heating rate of 10° C./min, (ii) by keeping at 120° C. for 5 minutes; (iii) by cooling to 0° C. at a cooling rate of 10° C./min and (iv) by reheating with a second measurement cycle to 200° C. at a heating rate of 10° C./min, the glass transition temperature being measured from the second measurement cycle.

58. The Kraft lignin nanoparticle according to claim 57, characterized in that the first glass transition temperature is ranging between 110° C. and 130° C. as determined by Differential Scanning Calorimetry; wherein the Differential Scanning Calorimetry is conducted (i) by heating the nanoparticle with a first measurement cycle from room temperature to 120° C. at a heating rate of 10° C./min, (ii) by keeping at 120° C. for 5 minutes; (iii) by cooling to 0° C. at a cooling rate of 10° C./min and (iv) by reheating with a second measurement cycle to 200° C. at a heating rate of 10° C./min, the glass transition temperature being measured from the second measurement cycle.

59. The Kraft lignin nanoparticle according to claim 54, characterized in that said nanoparticle has a three-dimensional structure that comprises at least two types of □-□ stacking, as determined by UV-Visible analysis.

60. The Kraft lignin nanoparticle according to claim 54, characterized in that said nanoparticle has a polydispersity index ranging between 0.05 and 0.20 as determined by Dynamic Light Scattering method.

61. The Kraft lignin nanoparticle according to claim 54, characterized in that said nanoparticle has Young’s modulus ranging between 1.0 GPa and 6.0 GPa as determined by Atomic Force Microscopy.
